(19) United States
(12) Patent Application Publication
(10) Pub. No.: US 2020/0292944 A1
(43) Pub. Date: Sep. 17, 2020

(54) METHOD OF MAKING A PATTERNED HYDROGEL AND KIT TO MAKE IT

(71) Applicant: Massachusetts Institute of Technology, Cambridge, MA (US)

(72) Inventors: Marianna Sofman, Cambridge, MA (US); Natasha Arora, Boston, MA (US); Linda Griffith, Cambridge, MA (US); Paula Hammond, Newton, MA (US)

(21) Appl. No.: 15/776,978

(22) PCT Filed: Nov. 17, 2016
(86) PCT No.: PCT/US16/62564

§ 371 Date: May 17, 2018

Related U.S. Application Data
(60) Provisional application No. 62/256,791, filed on Nov. 18, 2015.

(51) Int. Cl. G03F 7/34; G03F 7/00; C12M 1/32

(52) U.S. Cl. CPC G03F 7/343; C12M 23/12; G03F 7/346; G03F 7/0005

(57) ABSTRACT

Customizable hydrogel patterned into arrays of microwells, microchannels, or other microfeatures, and a process to prepare patterned hydrogel adherent to a solid support, are provided. A pattern of light is applied to photopolymerizable hydrogel precursor solution on a solid support, resulting in the formation of a patterned hydrogel bonded to the solid support. Hydrogel precursor solution is held by a ring of a customized height secured to a photomask or to a surface allowing transmission of a pattern of light. This process allows for facile customization and repeated fabrications of hydrogel microwells of desired heights and geometries with a micron or submicron scale resolution, adherent on various solid supports.

### CROSS-REFERENCE TO RELATED APPLICATIONS

This application claims benefit of U.S. Provisional Application No. 62/256,791, filed Nov. 18, 2015, which is hereby incorporated herein by reference in its entirety.

### STATEMENT REGARDING FEDERALLY SPONSORED RESEARCH OR DEVELOPMENT

This invention was made with Government support under grant no. CBET-0939511 awarded by the National Science Foundation under the Science and Technology Center Emergent Behaviors of Integrated Cellular Systems (EBICS). The Government has certain rights in the invention.

### FIELD OF THE INVENTION

The disclosed technology is generally in the field of hydrogel arrays, particularly in the methods of making and using hydrogel arrays with micron-scale geometries.

### BACKGROUND OF THE INVENTION

Micropatterned hydrogel presents an emerging platform for high-throughput drug discovery and clinical diagnostics on cells in both the two-dimensional (2D) and three-dimensional (3D) environments. Single cells, as well as homogenous or heterogeneous populations of small cell clusters (e.g., organoids), may be studied in hydrogel microarrays for emergent tissue behaviors in the development of new therapeutics and cell and gene therapies.

Soft lithography techniques utilize micro-molding tools to prepare hydrogel arrays. Molds (also called “stamps” or “stencils”) are generally made with polydimethylsiloxane (PDMS) and have micropatterned features such as micropillar arrays. Depending on whether these molds are brought into contact or placed above a substrate, precursor solution can be perfused and excluded from these regions, which upon curing forms a hydrogel microwell array with exposed floors separated by hydrogel walls (Heath D E, et al., Lab on a Chip, 15:2073-2089 (2015)) or with hydrogel as both the walls and the floors (Moeller H C, et al., Biomaterials, 29:752-763 (2008)).

Alternatively, molds can be applied to “ink” molecules with a certain surface property on the substrate in a pattern. For example, alkanethiols have been inked in array formats on gold surfaces to introduce differential wettability, which provides the features for forming hydrogel arrays (U.S. Patent Application No. US 2015/0293073).

However, hydrogel microwells prepared from this method may have several drawbacks. For example, delamination of the cured microwell and imperfect sealing with the substrate are common potential problems. The resolution of the geometries of these hydrogel microwells is also limited due to the swelling properties of elastomeric stamp materials. Residual stamp material may remain at the hydrogel interface and contaminate the non-fouling, bio-inert surfaces of some hydrogel microwells (Heath D E, et al., Lab on a Chip, 15:2073-2089 (2015)).

Photolithography is another technique for microfabricating patterns. For example, SU-8 master molds, commonly used to fabricate PDMS stencils, are prepared using this technique. Generally epoxy-based negative photoresist SU-8 is spincoated on a wafer, and UV irradiation through a photomask on SU-8 leaves behind a hardened resin in the shape of the transparent patterns of the photomask. (Jo B H, et al., J Microelectromechanical Syst, 9:76-81 (2000)). The approach to using photolithographic master molds to fabricate PDMS molds and subsequently using the PDMS molds to fabricate hydrogel microarrays is complicated and does not avoid the drawbacks of soft lithography mentioned above (U.S. Patent Application Publication No. US 2015/0293073). Alternatively, mask-directed photopolymerization of a liquid phase has been utilized to attempt providing patterned hydrogel. However, existing techniques rely on commercially available spacers (e.g., Teflon spacers) to define the thickness of hydrogel. These spacers tend to leak and have height restrictions (e.g., at least in millimeter or close to millimeter ranges) (U.S. Pat. No. 7,192,693). When spacers are omitted, the liquid phase only thinly coats the substrates (U.S. Pat. No. 6,686,161), which is likely to result in an uneven thickness of crosslinked hydrogel due to the curvature of liquid coating.

Therefore, it is an object of the invention to provide methods of preparing hydrogel microwell arrays of customizable geometries (e.g., height, width and length of a microwell, and spacing distance between microwells) that adhere to a substrate in a facile and reliable process.

It is another object of the invention to provide customizable hydrogel microwell arrays and their use in both cell culturing and microscopic imaging.

### SUMMARY OF THE INVENTION

Customizable hydrogel with patterns (e.g., microwells and microchannels) and a process to prepare patterned hydrogel adherent to a solid support are provided. The process photo-patterns hydrogel with a resolution at the micron scale (e.g., 1 μm) and adheres the patterned hydrogel on a solid support at the same time. This process eliminates the need to imprint patterns via soft lithography molds and circumvents the leakage problems or height restrictions with conventional spacers.

The formation of patterned hydrogel and its adhesion to a solid support are fabricated in the same step of exposing a hydrogel precursor solution to a pattern of light, where the solid support is placed on top and in contact with a hydrogel precursor solution that is contained within a boundary on a photomask. Generally, the hydrogel precursor solution fills up the volume defined by the height of a ring and the area enclosed by the ring that is secured onto a surface where a pattern of light can transmit. A ring provides a closed boundary of a certain height on top of the surface where a pattern of light can transmit, such that liquid of a certain volume can be retained therein without leakage. In some embodiments, the surface where a pattern of light can transmit is a photomask, so the ring is secured onto a photomask encircling some patterned regions on the photomask. In other embodiments, the surface of a secured ring is a clear surface to allow the transmission of a pattern of light created by a photomask. The photomask contains a custom-made design of patterns of clear and dark regions to allow transmission of a pattern of light. The height of the ring governs the volume of the precursor solution, and therefore the maximum depth of a formed patterned hydrogel. A solid support, which becomes adherent to final patterned hydrogel, is placed on top and in contact with the precursor solution. A pattern of light exposes the hydrogel precursor solution and the contacting solid support to induce formation of a patterned hydrogel that is covalently bonded with the solid support. Formed patterned hydrogel adherent to the solid support is lifted from the photomask and washed to remove unpatterned materials.

The process to fabricate a patterned hydrogel adherent to a solid support does not require any spacer to be secured to the solid support for the formation of patterned hydrogel of a certain height. The process does not require mechanical or chemical maneuvering to secure any spacer on the solid support, avoiding potential damage to the solid support. It also allows for repeated fabrications of many batches of patterned hydrogel on different solid supports. In embodiments where a pattern of light is created through a photomask and subsequently transmitted through a clear surface with a secured ring which holds a hydrogel precursor solution, fabrications of many different patterns of hydrogel on different solid supports can be realized with the process by utilizing photomasks with different pattern designs and solid supports of choice.

The patterned hydrogel may be an array of microwells, microchannels, or a combination thereof, where each micro-feature is independently in a circular, ovoid, quadrilateral, rectangular, square, triangular, pentagonal, hexagonal, heptagonal, or octagonal shape depending on the designs of a photomask to permit a pattern of light.

In some embodiments, the patterned hydrogel adherent on a solid support has the surface of the solid support as the floors/bottoms microwells or microchannels, i.e., not covered with the hydrogel, whereas hydrogel forms the side walls of microwells and/or microchannels. In other embodiments, the patterned hydrogel can be in the form of an array of micron- or millimeter-scale hemispherical bowls, wells, or channels where both the bottom and the side walls are hydrogel.

The patterned hydrogel is an array of microwells in the rectangular or circular shape with a diagonal distance or a diameter between about 30 μm and about 100 mm, preferably between 50 and 800 μm for certain applications such as small population cell culture, or between 3 mm and 100 mm for other applications. The height of the patterned hydrogel or the depth of void micro-features may be customized depending on applications, and is generally greater than 20 μm. The microwells or channels may be arranged in a variety of geometries. Neighboring microwells or microfeatures can be equally spaced from one another or irregularly spaced, generally separated by at least about 10 μm, more preferably at least about 20 μm, or by a gradient of wall thickness ranging from about 20 μm to about 100 μm.

The patterned hydrogel is covalently attached to a solid support including glass, film, silicon, ceramic, plastic, or an appropriate polymer such as (poly)tetrafluoroethylene, or (poly)vinylidenedifluoride. Preferably, the patterned hydrogel is covalently attached to a coverslip, a thin piece of glass, for cell culture and imaging under a microscope without disturbing the cultured cells.

Suitable hydrogel precursor materials have photopolymerizable functional groups, such as a methacrylate derivative, acrylate derivative, ethylene, diene, styrene, halogenated olefin, vinyl ester, acrylonitrile, acrylamide, n-vinyl pyrrolidone, and a polymer or block copolymer thereof. Exemplary hydrogel precursors include poly(ethylene glycol) dimethacrylate, poly(ethylene glycol) diacrylate, tetraethylene glycol diacrylate, ethylene glycol dimethacrylate, ethylene glycol diacrylate, dipropylene glycol dimethacrylate, dipropylene glycol diacrylate, and a polymer or block copolymer thereof.

Suitable solid support materials for the formed patterned hydrogel also have one or more photopolymerizable functional groups, such that during light exposure hydrogel is formed and bonded with the solid support. Photopolymerizable functional groups such as acrylate and methacrylate can be modified on the surface of a solid support. For example, silane coupling agents containing acrylate groups may be used to modify glass surfaces.

The patterned hydrogel on a solid support, particularly in an array of microwells and/or microchannels, is useful in a variety of medical and laboratory applications where an inexpensive and rapid patterning technique is required. Such applications include, but are not limited to, scaffolds for culturing and differentiating stem cells for use in tissue engineering and repair, drug delivery, angiogenic membranes, microfluidics, bioMEMs, coating, immobilization of biomolecules to surfaces with spatial fidelity, devices for microscale chromatography and electrophoresis, including biomolecules, applications in oligonucleotide arrays, proteomics, electrode arrays, and immobilization of cells and organisms.

A kit for making a patterned hydrogel adherent to a solid support is also provided, including a photomask with a pattern of clear and dark regions to allow transmission of a pattern of light, a ring attached or capable of being attached to the photomask providing a continuous boundary of at least 20 μm in height perpendicular to the photomask, optionally a hydrogel precursor solution with one or more solutes capable of being photopolymerized or the solutes and a diluent in separate containers, and optionally a solid support with photopolymerizable functional groups or agents to modify a solid support with photopolymerizable functional groups.

### BRIEF DESCRIPTION OF THE DRAWINGS

FIG. 1 shows a schematic representation of fabrication of hydrogel microwell array which is UV cured to a silanized glass coverslip via a patterned photomask. In the curing process, the silanized glass coverslip is placed directly on the SU-8 ring, creating a seal with the pre-polymer solution.

FIGS. 2A and 2B are drawings of representative designs on the photomask from Autocad files, in an array of a circular shape (2A) or an array of a square shape (2B).

FIG. 3 shows a bright-field image of hydrogel microwells of a well size of 600 μm by 600 μm, separated by 100 μm-thick wall.

FIGS. 4A-4F show bright-field images of customized hydrogel microwells. FIG. 4A shows a 400×400 μm well with a 50 μm-thick wall. FIG. 4B shows 400×400 μm wells with walls of a 20-100 μm thickness gradient. FIG. 4C shows 600×600 μm wells with 100 μm-thick walls. FIG. 4D shows 100×100 μm wells separated by 20 μm-thick walls. FIG. 4E shows 100×100 μm wells separated by 100 μm-thick walls. FIG. 4F shows wells of 400 μm in diameter seeded with human induced pluripotent stem cells (hiPSCs), separated by 100 μm-thick walls.

### DETAILED DESCRIPTION OF THE INVENTION

### I. Definitions

The term “hydrogel” refers to 3-D networks of molecules typically covalently (e.g., polymeric hydrogels) held together where water is the major component (usually greater than 80%).

The term “hydrogel array” or “hydrogel microarray” refers to a combination of two or more microlocations. Preferably an array is comprised of microlocations in addressable rows and columns. The thickness and dimensions of the polymer hydrogel and/or hydrogel arrays can vary dependent upon the particular needs of the user.

The term “microwell” here refers to a chamber or void in the hydrogel or surrounded by hydrogel. Each microwell typically has a volume of less than one milliliter, and is capable of holding and retaining a solid or liquid sample.

The term “photopatterned hydrogel” or “patterned hydrogel” refers to a hydrogel that is polymerized via irradiation, usually in the form of UV, containing features of specific geometries as defined by the clear and dark regions of a photomask through which irradiation occurs.

The term “polymerization” refers to the reaction by which monomer molecules combine to form polymer molecules.

The term “polymerizable group” refers to a group on the monomer or crosslinking agent that can link to another monomer or crosslinking agent to form a polymer. For example, the polymerizable group may be a methacrylate or an acrylate group, as described below.

The term “conversion” refers to the incorporation of polymerizable groups on the monomer and the crosslinking agent into a polymer (and subsequent hydrogel), which may be determined, for example, by monitoring the fraction of vinyl groups that have been incorporated into the polymer. “Essentially complete conversion” refers to an extent of conversion that is sufficient to form a patterned hydrogel, whereas “partial conversion” refers to an extent of conversion that is insufficient to form a patterned hydrogel and instead results in polymer molecules that remain soluble. Typically, “essentially complete conversion” represents between about 90% and about 100% conversion, and “partial conversion” represents between about 1-40% conversion.

The term “ring” refers to a continuous boundary of a certain height that may be deposited, mounted, or otherwise secured to a photomask or to a surface where a pattern of light transmits. The secured ring allows liquid of a defined volume and height to be held on the photomask or the surface where a pattern of light transmits. The ring may be in a circular, square, rectangular, or other shape, and may be made with various materials including photoresist.

The term “cytokine” refers to any cytokine or growth factor that can induce the differentiation of a hematopoietic stem cell to a hematopoietic progenitor or precursor cell and/or induce the proliferation thereof, and which may be linked to the hydrogel or included in the hydrogel according to the disclosure. Suitable cytokines for use in the method include, but are not limited to, erythropoietin, granulocyte-macrophage colony stimulating factor, granulocyte colony stimulating factor, macrophage colony stimulating factor, thrombopoietin, stem cell factor, interleukin-1, interleukin-2, interleukin-3, interleukin-6, interleukin-7, interleukin-15, Flt3L, leukemia inhibitory factor, insulin-like growth factor, and insulin. The term “cytokine” as used herein further refers to any natural cytokine or growth factor as isolated from an animal or human tissue, and any fragment or derivative thereof that retains biological activity of the original parent cytokine. The cytokine or growth factor may further be a recombinant cytokine or a growth factor such as, for example, recombinant insulin. The term “cytokine” as used herein further includes species-specific cytokines that while belonging to a structurally and functionally related group of cytokines, will have biological activity restricted to one animal species or group of taxonomically related species, or have reduced biological effect in other species. The term “cytokine” as used herein further includes “morphogen”, which refers to a substance governing the pattern of tissue development and, in particular, the positions of the various specialized cell types within a tissue. It spreads from a localized source and forms a concentration gradient across a developing tissue. In developmental biology a morphogen is rigorously used to mean a signaling molecule that acts directly on cells (not through serial induction) to produce specific cellular responses dependent on morphogen concentration. Well-known morphogens include, but are not limited to, transforming growth factor beta (TGF-β), Hedgehog/Sonic Hedgehog, Wingless/Wnt, epidermal growth factor (EGF), and fibroblast growth factor (FGF). Morphogens are defined conceptually, not chemically, so simple chemicals such as retinoic acid may also act as morphogens.

The term “cell or population of cells” as used herein refers to an isolated cell or plurality of cells excised from a tissue or grown in vitro by tissue culture techniques. In the alternative, a population of cells may also be a plurality of cells in vivo in a tissue of an animal or human host.

### II. Composition

A patterned hydrogel adherent to a solid support may contain features of an array of microwells in combination with one or more additional microfluidic features, such as one or more micro-channels. A patterned hydrogel adherent to a solid support may contain exclusively microwells.

The hydrogel array of micro-features may be formed in a variety of shapes and dimensions as desired for particular applications. Generally, the patterned hydrogel is formed to possess a solid bottom, one or more hydrogel side walls, and an opening located on the surface of the patterned hydrogel.

Alternatively, the patterned hydrogel can be in the form of an array of micron- or millimeter-scale hemispherical bowls, wells, or channels where the bottom and the side walls are hydrogel. These forms where the solid support adhering to the hydrogel is not exposed as the solid floor/bottom are typically made with additional microfeatures on a photomask within the boundary encircled by a mold attached thereto, where the microfeatures are generally clear to allow transmission of light and have a shape of the desired voids in the final hydrogel array and a height not greater than that of the mold which defines the boundary to retain liquid on the photomask.

The hydrogel microwells can have any suitable shape. For example, the microwells can be circular, ovoid, quadrilateral, rectangular, square, triangular, pentagonal, hexagonal, heptagonal, or octagonal. In some embodiments, the microwells are rectangular in shape. In these instances, the shape of the microwells can be defined in terms of the length of the four side walls forming the perimeter of the rectangular microwell.

In some embodiments, the microwells are spherical in shape. In certain embodiments, the microwells are circular and have a diameter of between 3 and 100 mm, more preferably between 5 and 80 mm. For certain applications, the microwells are an array of circular wells, each having a diameter of between 30 μm and 1000 μm, more preferably between 50 and 800 μm.

The depth of the microwells, governed by the height of the mold providing a closed boundary perpendicular to the plane defined by the photomask, can be modified to provide microwells having the desired volume and/or volume-to-surface-area ratio for particular applications. In certain instances, the depth of the microwells ranges from about 20 microns to about 1.5 mm, more preferably from about 50 microns to about 500 microns, most preferably from about 100 to about 500 microns.

The microwells or channels may be arranged in a variety of geometries depending upon the overall shape of the patterned hydrogel. For example, in some embodiments, the microwells are arranged in rectangular or circular arrays. In the case of hydrogel microwell “plates” containing a plurality of microwells, the microwells may be equally spaced from one another or irregularly spaced. In preferred embodiments, the edges of neighboring microwells are separated by at least about 10 μm, more preferably at least about 20 μm, or by a gradient of wall thickness ranging from about 20 μm to about 100 μm. In certain embodiments, the edges of neighboring microwells are separated by at least about 20 μm, 50 μm, 100 μm, about 200 μm, about 300 μm, or about 400 μm.
In particular embodiments, the patterned hydrogel contain an array of microwells arranged in a 2:3 rectangular matrix, to form a microwell plate (also known as a microtiter plate). In some cases, the microwell microfluidic device has a total of six, 24, 96, 384, 1536, 3456, or 9600 microwells arranged in a 2:3 rectangular matrix.

In certain embodiments, the patterned hydrogel has microfeatures (e.g., wells) of one or more dimensions, including well diameter, well spacing, well depth, well placement, plate dimensions, plate rigidity, and combinations thereof, equivalent to the standard dimensions for microwell plates published by the American National Standards Institute (ANSI) on behalf of the Society for Biomolecular Sciences (SBS). In this way, the patterned hydrogel adherent on a solid support can be rendered compatible with existing technologies for plastic microtiter plates, including 8-channel micropipettes and automated plate readers.

1. Precursor to Make Hydrogel

Generally a solution containing polymers with at least two polymerizable groups can be photocrosslinked to form hydrogel. The at least two polymerizable groups can be identical or different. Additional crosslinkers can be optionally added to the polymer solution to provide features such as degradability. Alternatively, a solution containing monomers, crosslinkers, and photoinitiators is photo-polymerized to form hydrogel microarrays. The polymerizable group on the monomer, polymer, crosslinking agent, or other precursor materials to make photopatternable hydrogel include a methacrylate group, an acrylate group, vinyl groups, aryl azides, azido-methyl-coumarins, benzophenones, anthraquinones, certain diazo compounds, diazirines, and psoralen derivatives.

Exemplary monomer materials include, but are not limited to, methacrylate derivatives, acrylate derivatives, ethylenes, dienes, styrenes, halogenated olefins, vinyl esters, acrylonitriles, acrylamides, n-vinyl pyrrolidones, and mixtures thereof. Suitable methacrylate derivatives include, but are not limited to, 2-hydroxyethyl methacrylates, methyl methacrylates, methacrylic acids, n-butyl methacrylates, glycidyl methacrylates, n-propyl methacrylates, poly(ethylene glycol) monomethacrylates, and mixtures thereof. Suitable acrylate derivatives include, but are not limited to, 2-hydroxyethyl acrylate, 2-methoxyethyl acrylate, acrylic acid, n-butyl acrylate, glycidyl acrylate, n-propyl acrylate, poly(ethylene glycol) monoacrylate, and mixtures thereof.

Exemplary crosslinkable polymers or crosslinking agents include, but are not limited to, tetraethylene glycol dimethacrylate, tetraethylene glycol diacrylate, ethylene glycol dimethacrylate, ethylene glycol diacrylate, alkylene glycol diacrylate, dipropylene glycol dimethacrylate, dipropylene glycol diacrylate, poly(ethylene glycol)dimethacrylate, poly(ethylene glycol)diacrylate, poly(alkylene glycol) diacrylate, bisacrylamide, and mixtures thereof. Generally, poly(akylene glycol)-based crosslinkable polymers generate bio-inert, non-fouling hydrogel microarray. Some embodiments provide hydrogel microarray made with natural polymers and other biologically active molecules that are functionalized with polymerizable groups. These natural polymers or biologically active molecules include hyaluronic acid, elastin, collagens, and alginic acid.

Exemplary photoinitiators include 2,2-dimethoxy acetophenone, 1-hydroxycyclohexyl phenyl ketone, 2-methyl-1-[4-(methylthio)phenyl]-2-(4-morpholinyl)-1-propoanone, 2-hydroxy-1-[4-(hydroxyethoxy)phenyl]-2-methyl-1-propanone, photo and mixtures thereof. Other photoinitiators include benzophenones, xanthones, and quinones (each of which typically require an amine co-synergist); benzoin ethers, acetophenones, benzoyl oximes, and acylphosphines (each of which typically do not require a co-initiator such as an aliphatic amine); as well as other known photoinitiators, e.g., TEMED (N,N,N′,N′-tetramethylethylendiamine). Suitable photoinitiators include those from the α-hydroxyketone family (e.g., IRGACURE®184, DAROCUR®1173, IRGACURE®127, IRGACURE®2959, and IRGACURE®500), the phenylglyoxylate family (e.g., IRGACURE®754, Darocur®MBF), the acylphosphine oxide family (e.g., LUCIRIN®TPO, LUCIRIN®TPO-L, IRGACURE®2100, IRGACURE®819, IRGACURE®819-DW, DAROCUR®4265, IRGACURE®2022), the α-aminoketone family (e.g., IRGACURE®07, IRGACURE®369, IRGACURE®379, AND IRGACURE®389), the benzophenone family (e.g., DAROCUR®BP), benzildimethyl ketal family (e.g., IRGACURe®651), the sulfonium salt family (e.g., IRGACURE®250, IRGACURE®270, IRGACURE®PAG 290, AND IRGACURE®GSID26-1), IRGACURE®784, the oxime ester family (e.g., IRGACURE®OXE 01 AND IRGACURE®OXE 02), and the photoacid generator family (e.g., IRGACURE®PAG 103, IRGACURE®PAG 121, IRGACURE® PAG 203, IRGACURE®CGI 1380, IRGACURE®CGI 1907, and IRGACURE®CGI 725), all available from vendors including BASF. The amount by weight of initiator (e.g., photoinitiator or other initiator) generally ranges from 0% to about 5.0%.

Some embodiments provide microarrays formed with a degradable, photopatterned hydrogel. Suitable crosslinking agents for forming degradable, photopatterned hydrogel microarrays include hydrolytically degradable crosslinking agents and enzymatically degradable crosslinking agents. Exemplary degradable crosslinking agents include, but are not limited to, poly(ε-caprolactone)-b-tetraethylene glycol-b-poly(ε-caprolactone)dimethacrylate, poly(ε-caprolactone)-b-poly(ethylene glycol)-b-poly(ε-caprolactone)dimethacrylate, poly(lactic acid)-b-tetraethylene glycol-b-poly(lactic acid)dimetbacrylate, poly(lactic acid)-b-poly(ethylene glycol)-b-poly(lactic acid)dimethacrylate, poly(glycolic acid)-b-tetraethylene glycol-b-poly(glycolic acid)dimethacrylate, poly(glycolic acid)-b-poly(ethylene glycol)-b-poly(glycolic acid)dimethacrylate, poly(ε-caprolactone)-b-tetraethylene glycol-b-poly(ε-caprolactone)diacrylate, poly(ε-caprolactone)-b-poly(ethylene glycol)-b-poly(ε-caprolactone)diacrylate, poly(lactic acid)-b-tetraethylene glycol-b-poly(lactic acid)diacrylate, poly(lactic acid)-b-poly(ethylene glycol)-b-poly(lactic acid)diacrylate, poly(glycolic acid)-b-tetraethylene glycol-b-poly(glycolic acid) diacrylate, poly(glycolic acid)-b-poly(ethylene glycol)-b-poly(glycolic acid) diacrylate, and mixtures thereof.

Some embodiments provide microarrays formed with porous, photopatterned hydrogel. Suitable porogens include, but are not limited to, beads (such as PMMA beads or polystyrene beads), salts, sugars, and waxes. The pore sizes of the porous patterned hydrogels may be varied simply through the choice of porogen diameter.

2. Optional Modification of Hydrogel Microarray

Microarrays such as microwells may further include covalently or non-covalently immobilized biologically active molecules coated on the surface of microwells. For example, to improve cell attachment and performance on hydrogel microwells, especially on those with the microwell floor exposed and only the microwell wall made with photocrosslinked hydrogel, a variety of biological materials may be used to coat the surface of microwells, including extracellular matrix such as MATRIGEL® (available from vendors such as Corning), attachment and adhesion proteins such as collagen, laminin and fibronectin, basic synthetic polymers such as poly-D-lysine (PDL), and mucopolysaccharides such as heparin sulfate, hyaluronidate and chondroitin sulfate, both individually and as mixtures.

The hydrogel microarray may be readily adapted to immobilize covalently or non-covalently a variety of therapeutic, prophylactic and diagnostic agents. These agents may be introduced into the patterned hydrogels by forming the hydrogels in the presence of the biologically active molecules, by allowing the biologically active molecules to diffuse into the patterned hydrogels, or by otherwise introducing the biologically active molecules into the patterned hydrogels.

Therapeutic, prophylactic and diagnostic agents may be proteins, peptides, sugars or polysaccharides, lipids or lipoproteins or lipopolysaccharids, nucleic acids (DNA, RNA, siRNA, miRNA, tRNA, piRNA, etc.), or small molecules (typically 2000 D or less, more typically 1000 D or less, organic, inorganic, natural or synthetic). For example, biologically active molecules can be encapsulated, entrapped or immobilized in the hydrogel microarray, such as collagens of all types, elastin, hyaluronic acid, alginic acid, desmin, versican, matricelluar proteins such as SPARC (osteonectin), osteopontin, thrombospondin 1 and 2, fibrin, fibronectin, vitronectin, albumin, prior to photo-polymerization of hydrogel or after hydrogel array is formed. Useful functional groups can be inserted into the hydrogel to covalently immobilize one or more therapeutic, prophylactic, or diagnostic agents. A photo-reactive functional group (e.g., vinyl groups, aryl azides, azido-methyl-coumarins, benzophenones, anthraquinones, certain diazo compounds, diazirines, and psoralen derivatives) present on polymer molecules within the patterned hydrogel formed can be used to covalently attach agents to the hydrogels.

In some embodiments, the hydrogel microwell array includes agents for enhancing cell survival, proliferation or migration, maintaining stem cell pluripotency, or promoting differentiation of stem cells. For example, human pluripotent stem cells, including human embryonic stem cells and induced pluripotent stem cells, may be cultured and differentiated in the wells of the hydrogel microarray. Suitable agents to add to the hydrogel microwells include biological and chemical agents for assaying, maintaining, and differentiating human pluripotent stem cells. Exemplary agents include grow factors such as basic fibroblast growth factor (bFGF), bone morphogenetic protein 4 (BMP4), Activin A, insulin, transferrin, selenium, FGF7, nicotinamide, islet neogenesis associated peptide (INGAP) and exendin-4, a long acting GLP-1 agonist, or a mixture thereof. These agents can be released into the cell culture in a controlled manner by modulating the immobilization and/or degradation of the linkage of agents to the hydrogel.

2. Solid Support

Preferably, the hydrogel microarray is covalently attached onto a solid support or a substrate, during the fabrication process of photopatterned hydrogel microarrays. The solid support, or the substrate, generally provides the bottom (i.e., floors) of any formed hydrogel microarrays, and is preferably thin and clear (e.g., a coverslip) for imaging purposes. For other applications, the substrate material, thickness, and flexibility may vary.

Suitable substrate (i.e., solid support for the formed hydrogel microwells) includes, but is not limited to, film, glass, silicon, modified silicon, ceramic, plastic, or any type of appropriate polymer such as (poly)tetrafluoroethylene, or (poly)vinylidenedifluoride. A preferred substrate is glass, preferably of a thickness such as a microscope slide coverslip. Preferably the solid support is a material such as nylon, polystyrene, glass, latex, polypropylene, or activated cellulose. The solid support may be present in a form such as a slide, bead, membrane, microwell, or centrifuge tube.

Generally a coupling agent is attached onto the surface (e.g., glass) of the substrate, such that photopolymerizable groups are presented on the surface of the substrate and can photochemically link with the hydrogel precursor material. Appropriate treatment of the substrate provides adherence of hydrogel to the substrate, e.g., with γ-methacryl-oxypropyl-trimethoxysilane (“Bind Silane”, Pharmacia). For example, acrylate groups can be modified onto glass substrates via a silane coupling agent, e.g., (3-acryloxypropyl)trimethoxysilane. The coupling reaction between the hydrogel precursor and the substrate can also take place via photo cycloaddition known in the art.

3. Photomask with a Ring or a Clear Surface with a Ring

Photopatterning of liquid phase polymerizations occurs using a photomask where a pattern is defined by the clear and dark regions. The initiating light penetrates through the clear region of the mask, to the solution, where it initiates the polymerization reaction. The dark regions block the initiating light and prevent the polymerization reaction.

In some embodiments, the photomask is fabricated to have patterns defined by clear and dark regions and to have a ring secured on its surface. In other embodiments, the photomask with patterns of clear and dark regions is placed before a surface that allows transmission of light, and the surface has a ring secured thereto. If the photomask or the surface to transmit light defines a plane with x- and y-axes, the ring has an enclosed area and a z-axis height that governs the volume of the precursor solution and therefore the depth of the hydrogel arrays. Precursor solution is added to fill up the space defined by the ring on the photomask. A surface-treated coverslip, film, or other solid support for the ultimate hydrogel array is placed on top to seal the precursor solution, which under irradiation photo-polymerizes and covalently links with the coverslip, film, or other solid support in the regions exposed to a pattern of light.

Generally the pattern information is created in a drawing package, often in AutoCAD or other suitable software packages such as L-Edit. The data is processed into internal CAD format (Gerber) and transferred to a lithography tool which then exposes the design onto the photomask. The photomasks can be glass or film photomasks. For example, in order to create hydrogel microarrays having microwells separated by hydrogel walls, the pattern on photomask will have clear regions outlining the wall of hydrogel microwells to be made. Each microwell or microlocation generally is about 1 μm to about 1 millimeter in size (e.g., diameter or width and length) to generate micron-sized location, although the size can go up to a few millimeters or centimeters depending on the user's applications.

A ring can be a mold that provides a continuous boundary, which is deposited, mounted, or otherwise attached to the photomask to surround some photomask patterns or attached to a surface that transmits light and is placed after a photomask. The ring may be in a circular, square, rectangular, or other shape, and it may or may not define the final periphery of the formed patterned hydrogel depending on the pattern of light. The height of the ring defines the depth of the precursor solution and therefore the height of the formed hydrogel microarrays. The ring can be made with a material that does not interfere with the photo-crosslinking of hydrogel microwells. The ring can be custom-made with photoresist such as epoxy-based polymers like SU-8 (with the presence of 8 epoxy groups) or other polymers of a wanted diameter, thickness, and height. Suitable photoresist materials include light-sensitive materials such as poly(methyl methacrylate), poly(methyl glutarimide), phenol formaldehyde resin, SU-8, and off-stoichiometry thiol-enes (OSTE) polymers. These photoresist materials after exposure and development leaves behind a ring that at least fairly adheres to the photomask, such that a precursor solution will be contained within the its boundary on the photomask. Alternatively, TEFLON® rings of a defined height providing a continuous boundary can be glued or otherwise attached to the photomask, such that precursor solution can be contained therein.

4. Kit

Some embodiments provide one or more photomasks with desired pattern designs, one or more molds providing a closed boundary of one or more heights, one or more precursor solution in a lyophilized dry form or in the liquid form, and optionally one or more solid supports with surface modification for photocrosslinks, can be included in a kit. Users may assemble the molds on top of the photomasks by means such as gluing, or the kit provides molds mounted on the photomasks, such that one or more hydrogel arrays having micro-features defined by the patterns on the photomasks and a height defined by the mold can be fabricated and adhered to the solid support with a light source.

### III. Method of Making

1. Design of Patterns and Securing a Ring

Generally the pattern information is created in a layout editor (e.g., AUTOCARD®, SOLIDEDGE, ADOBE® ILLUSTRATOR, CLEWIN, WIEWEB Inc.) or standard computer aided drafting software. The data may be processed into internal CAD format (Gerber) and transferred to a lithography tool which then exposes the design onto the photomask. The photomasks can be glass or film photomasks. For example, in order to create hydrogel microarrays having microwells separated by hydrogel walls, the pattern on photomask will have clear regions outlining the wall of hydrogel microwells to be made. Each microwell or microlocation generally is about 1 μm to about 1 millimeter in size (e.g., diameter or width and length) to generate micron-sized location, although the size can go up to a few millimeters or centimeters depending on the user's applications. The resolution of the microfeature is generally limited by the pixel size given by the imaging system that exposes the design onto photomasks. For example, the pixel size may be in the sub-micron range, e.g., 0.8 μm, and therefore the design on the photomask should have a microfeature greater than the pixel size.

The ring defines the depth of the precursor solution and therefore the height of the formed hydrogel microarrays. It can be deposited, mounted, or attached to the photomask, such that it creates a closed boundary of a wanted height in the direction generally perpendicular to the plane defined by the photomask and encircles some patterned regions on the photomask. The ring can also be secured to a surface that transmits light, and the surface is placed after a photomask in the light path such that a pattern of light transmits through the surface where a ring is secured to.

An exemplary method to customize the size, thickness, and height of a ring includes spin coating photoresist of a wanted height on the photomask and exposing the photoresist to a light source to harden and develop into a continuous boundary of any shape. Spin coating techniques generally includes putting photoresist on the photomask that is placed on a rotating stage. The rotation speed, the acceleration, and the photoresist viscosity will define the thickness of the photoresist layer, e.g., about 20 μm, 50 μm, 100 μm, or more. Subsequently, the photoresist is soft baked to evaporate solvent to make the photoresist more solid, and optionally any irregularities on the edge due to surface tension are removed. Exposing the photoresist to a light source through a photomask changes the local properties of the photoresist. Developing the photoresist in the wanted geometry provides a continuous boundary in the direction generally perpendicular to the plane defined by the photomask.

Alternatively, existing ring molds of a wanted height (e.g., TEFLON® rings) can be glued or otherwise attached to the photomask or the surface where a pattern of light transmits, such that precursor solution can be retained therein.

2. Surface Treatment of Substrate/Solid Support

Photopatterned hydrogel microarrays can be covalently attached to substrate in the same fabrication process of the microarrays. Generally the substrate or solid support is pre-treated on the surface to contain photopolymerizable functional groups, unless the substrate has such groups on the surface to begin with. A coupling agent is attached onto the surface (e.g., glass) of the substrate, such that photopolymerizable groups are presented on the surface of the substrate and can photo-chemically link with the hydrogel precursor material. Appropriate treatment of the substrate provides adherence of hydrogel to the substrate, e.g., with γ-methacryl-oxypropyl-trimethoxysilane (“BIND SILANE”, Pharmacia). For example, acrylate groups can be modified onto glass substrates via a silane coupling agent, e.g., (3-acryloxypropyl)trimethoxysilane. The coupling reaction between the hydrogel precursor and the substrate can also take place via photo cycloaddition known in the art.

3. Assembly of Components and Hydrogel Microarray Formation.

The hydrogel microarray may have a thickness of less than 1 millimeter, desirably a thickness of between about 1 and about 500 microns, and even more preferably a thickness of between about 20 and about 100 microns. Desirably the hydrogel array contains microlocations that are each from about 1 to about 800 microns in size, particularly from about 50 to about 600 microns, and especially about 100, 200, 300, 400, 500, or 600 microns in various shapes desired by users.

Precursor solution generally fills up the space defined the enclosed area and the height of a ring that is secured to a surface that transmits a pattern of light. Generally a solid support is placed directly on top of the ring, creating a seal between the precursor solution and the solid support. A light source is placed generally along the axis perpendicular to the photomask (e.g., under the photomask), irradiating light that penetrates designed regions of the photomask—resulting in a pattern of light—and exposing the precursor solution and the solid support for a period of time and under suitable conditions. This exposure induces essentially complete conversion of polymerizable groups on the monomer and the crosslinking agent, or essentially complete crosslinks of polymers having at least two functional groups to form crosslinked hydrogel in patterned regions. The formed hydrogel is also covalently linked to the surface-treated substrate during the exposure process. For a given polymerization solution, there generally exists an optimal light intensity that results in the shortest exposure times required to convert the liquid solution to a crosslinked hydrogel, whereas light intensities that are higher or lower require longer exposure times. As a result, a patterned material can be generated from the precursor solution by effectively modulating the light intensity, for example, through the opacity of a photomask. In essence, for a given exposure time, the regions that receive the optimal light intensity reach complete conversion (and thus form a highly crosslinked hydrogel) while regions receiving less than optimal light intensity (e.g., high light intensities) result in a partially polymerized, yet not completely crosslinked, solution that can be washed away.

Formed hydrogel microarray adhering to the substrate or solid support is lifted away from the photomask. It can be cleaned and stored in a moisturized environment or in ethanol to retain the structure and sterility. Other methods to sterilize formed hydrogel microarray include steam sterilization, gamma sterilization, immersion in antibacterial solutions, and other means known in the art.

In some embodiments, the fabrication may further include coating or immersing the patterned hydrogel on a solid support with one or more agents to modify the surface properties of the microwells. For example, the stiffness, cell-adhesion propensity, hydrophobicity, and/or optical properties of the patterned hydrogel including its solid support may be modified.

### III. Method of Using

The patterned hydrogel can be used in a variety of medical applications where an inexpensive and rapid patterning technique is required. Such applications include, but are not limited to, scaffolds for culturing and differentiating stem cells for use in tissue engineering and repair, drug delivery, angiogenic membranes, microfluidics, bioMEMs, coating, immobilization of biomolecules to surfaces with spatial fidelity, devices for microscale chromatography and electrophoresis, including biomolecules, applications in oligonucleotide arrays, proteomics, electrode arrays, and immobilization of cells and organisms. The macroscopic properties of these materials can readily be controlled to function in a variety of different applications. For example, simple variations in the precursor solution, such as an increase in the crosslinker concentration, will result in increased gel moduli or slower drug delivery. Channels of varying size can be patterned into the hydrogel for use as microfluidic devices. Porous hydrogels can be patterned and used in chip arrays.

1. Cell Culture and Formation of Tissue Clusters

The method to prepare patterned hydrogel allows for customization of the hydrogel to suit the specific culturing platform needs, by allowing for the modification of culture area, shape, hydrogel wall height, and biofunctionalization of the hydrogel material. Modular feature of the patterned hydrogel array designs also allows for the precise manipulation, arrangement, and distribution of the growing tissue clusters.

In a preferred embodiment, the solid support adherent to the patterned hydrogel is glass of a thin thickness to allow for high resolution imaging under microscope.

The patterned hydrogel may be used for long-term cell or tissue culture with high integrity, even at different temperature and with post-fabrication washes as needed in cell cultures. They can be used to study cell survival, proliferation, and migration in long-term culture.

In some embodiments, patterned hydrogel in the form of arrays of microwells or microchannels are used to culture budding pre-organoids and perform high-throughput imaging of budding pre-organoids. With to the micron-scale features of microwells, smallest-budding unit may be cultured in large quantities. Organoids, pre-organoids, and spheroids refer to the self-organization of mammalian cells into 3D cultures from primary tissue or from embryonic stem cells (ESCs) or induced pluripotent stem cells (iPSCs) (Fatehullah A, et al., Nature Cell Biology, 18:246-254 (2016)). They are generally a well-defined, stable culture system capable of sustaining the long-term growth of near-physiological cells. These organoids or pre-organoids show great promise in faithfully recapitulating the in vivo tissue architecture and containing the full complement of stem, progenitor and differentiated cell types.

For example to model lingual carcinoma disease, mammalian lingual stem cells can be cultured in the pattern hydrogel microwells to form spherical and budding organoids, for assaying tamoxifen inductions, lineage tracing, and engrafhnent studies. Similarly, cells from circumvallate tissue can be cultured in the pattern hydrogel microwells to form spherical and budding organoids for functional assays, gene expression analysis, and cell cycle analysis. Cells from salivary gland can be cultured in the pattern hydrogel microwells to form ductal branching organoids and lobular spherical organoids to model the disorder of hyposalivation, and be analyzed for gene expression and transplantation studies. Cells from oesophageal tissue can be cultured in the pattern hydrogel microwells to form spherical multilayered organoids or budding morphology. Cells form adult stomach, intestine, colon, liver, pancreas, prostate, lung, retina, inner ear, brain, or kidney, or from ESCs or iPSCs can be cultured in the pattern hydrogel microwells to form spherical organoids or budding organoids to model diseases and disorders in the related tissues.

2. High-Throughput or Micro-Scale Sample Analysis

The patterned hydrogel adherent on a solid support in the form of an array of microwells or micro-channels can be used to analyze one or more fluid samples. In certain embodiments, the patterned hydrogel microarrays are used to detect a variety of analytes based of the design of the microfluidic device, including small molecules, proteins, lipids, polysaccharides, nucleic acids, prokaryotic cells, eukaryotic cells, particles, viruses, metal ions, and combinations thereof.

In some cases, the patterned hydrogel microarrays are used to conduct point-of-care diagnostic testing. In these embodiments, the patterned hydrogel microarrays can be designed to operate without any supporting equipment, such as personal computers, pumps, or external instrumentation. For example, the patterned hydrogel microarrays may contain one or more assay regions containing one or more assay reagents selected so as to provide a response that is visible to the naked eye. In some cases, the assay reagent can be an indicator that exhibits colorimetric and/or fluorometric response in the presence of the analyte of interest. Indicators may include molecules that become colored in the presence of the analyte, change color in the presence of the analyte, or emit fluorescence, phosphorescence, or luminescence in the presence of the analyte. In these embodiments, the presence of an analyte may be ascertained by simple visual examination, optionally under a blacklight. In some cases, the quantity of one or more analytes may be determined by visual inspection of the color or fluorescence of an assay region, for example, by comparison to known colors at predetermined analyte concentrations. Alternatively, a portable device, such as a digital camera, flatbed scanner, or cellular phone may be used to analyze the response of the analyte region.

In other embodiments, the patterned hydrogel microarrays may be used in conjunction with external instrumentation. For example, a patterned hydrogel microarray may contain one or more fluid outlets that connect the patterned network to one or more external instruments, such as a mass spectrometer, fluorometer, UV-Vis spectrometer, IR spectrometer, gas chromatograph, gel permeation chromatograph, DNA sequencer, Coulter counter, or combinations thereof, that can be used to analyze the fluid flowing from the patterned network. It is appreciated by one skilled in the art that while a closed boundary is utilized in order to retain precursor solution during light exposure and results in a leak-proof patterned hydrogel, a cut, truncate, drilling, or other means to creating an opening in the patterned hydrogel may be performed to connect the patterned network to an external instrument. The patterned hydrogel microarrays may also contain one or more assay reagents are selected to facilitate radiological, magnetic, optical, and/or electrical measurements used to identify and/or quantify one or more analytes in a liquid sample.

The patterned hydrogel adherent on a solid support can be used to analyze a variety of biological fluids, including blood, urine, plasma, serum, tears, lymph, bile, cerebrospinal fluid, interstitial fluid, aqueous or vitreous humor, colostrum, sputum, amniotic fluid, saliva, anal and vaginal secretions, perspiration, semen, transudate, exudate, and synovial fluid. In certain embodiments, the patterned hydrogel in the form of a microfluidic device is used to perform a lateral flow-type immunoassay, for example, to detect pregnancy, fertility, narcotics, HIV, Troponin T, malaria, Avian Flu, respiratory diseases, sickle cell anemia, or combinations thereof. Samples to be assayed in the patterned hydrogel include suspended particles or large molecules, such as blood, environmental slurries, multi-phase suspensions, and other raw biological samples such as samples containing large macromolecules (such as DNA, RNA, and combinations thereof), suspended cells, or viruses. These assays can be used to identify and/or quantify a pathogen, such as a bacteria, protest, or virus, in a biological sample. The patterned hydrogel microarrays may also be useful for performing and/or optimizing polymerase chain reactions (PCRs).

The patterned hydrogel arrays can be used to analyze environmental samples, including water and soil samples, for example, to detect or quantify one or more heavy metals within a sample. They can also be used in quality control applications, including the analysis of food samples and pharmaceutical products.

Patterned hydrogel arrays may also be used in controlled crystal engineering. For example, they can be used to selectively prepare desirable polymorphs of pharmaceuticals. They can also be used to determine optimal conditions for protein crystallization.

Patterned hydrogel arrays may also be used to separate and/or purify samples, including complex biological samples. Electrophoresis can be performed within patterned hydrogel microchannels to separate ionic species, including biomolecules. Patterned hydrogel arrays may also be used in chromatographic separations (e.g., protein fractionation), for example, by filling a micro-channel with a size exclusion or ion exchange resin.

3. Modification for Implantable Devices

The biocompatible patterned hydrogels may also be applied to, or formed on, any implantable medical device, including, but not limited to, chemical sensors or biosensors (such as devices for the detection of analyte concentrations in a biological sample), cell transplantation devices, drug delivery devices such as controlled drug-release systems, electrical signal delivering or measuring devices, prosthetic devices, and artificial organs. The hydrogel improves the biocompatibility of the implanted medical device (such as the biocompatibility and communication of neuroelectrodes and pacemaker leads with surrounding tissues), improves the sealing of skin to percutaneous devices (such as in-dwelling catheters or trans-cutaneous glucose sensors), enhances tissue integration, and provides barriers for immunoisolation of cells in artificial organs systems (such as pancreatic cells devices), and improves the healing of vessels after balloon angioplasty and stent placement. The patterned hydrogels may be immobilized onto (or within) a surface of an implantable or attachable medical device body.

### EXAMPLES

### Example 1. Fabrication of Hydrogel Microwell Array

Materials and Methods

A photomask, designed using Autocad, contained the customized microwell dimensions and shapes. SU-8, an epoxy-based negative photoresist, was spin-coated, exposed, and developed in the shape of a ring on the photomask using standard photolithography techniques, encircling the 2-D designs of the photomask. The height of the final microwells correlated to the height of the SU-8 ring, which was controlled by modifying the spincoating parameters.

Silanization chemistry was performed on coverslips to allow for the bonding of the free acrylate groups of the PEG-based hydrogel to the glass coverslip. Specifically, prior to silanization, the glass coverslips were cleaned through a series of sonication steps with micro 90 soap, deionized water, 1M NaOH, and ethanol, followed by drying with nitrogen gas. Following oxygen plasma treatment, the coverslips were immersed in a solution of (3-acryloxypropyl)trimethoxysilane. The silane portion reacted with activated hydroxyl groups on the surface of glass (in this Example, coverslip or cover glass), therefore activing as a coupling agent by functionalizing acrylate groups on the coverslip, which were then reactive with the hydrogel precursor solution.

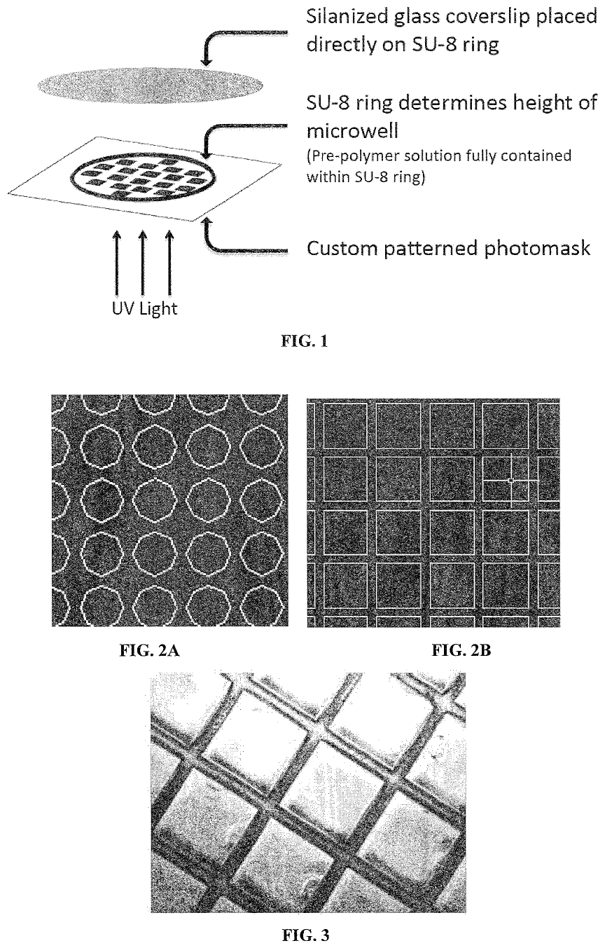

PEG-diacrylate (PEGDA) solution was pipetted into the SU-8 ring on the surface of the photomask to fill and be fully contained therein. The functionalized coverslip was placed directly on top of the SU-8 ring, being in contact and creating a seal with the PEGDA solution. A UV source was aligned parallel with the photomask. UV light was irradiated from under the photomask, as shown in FIG. 1, for 20 seconds at an intensity of 200 mW/cm². The PEGDA solution that was exposed to the UV light became crosslinked among the PEGDA molecules and with the acrylate functional group on the coverslip. Unexposed regions that were blocked by the photomask were dissolved with an ethanol wash. The final hydrogel microwell array adherent on the coverslips was stored at 4° C. in ethanol until further use.

Results

FIGS. 2A and 2B show different designs of the photomask from Autocad files. FIG. 3 shows a bright-field image of a hydrogel array of 600-μm-wide-by-600-μm-long wells separated by 100 μm thick walls, where the floor of microwells was the coverslip and the wall of microwells was the hydrogel covalently attached to the coverslip.

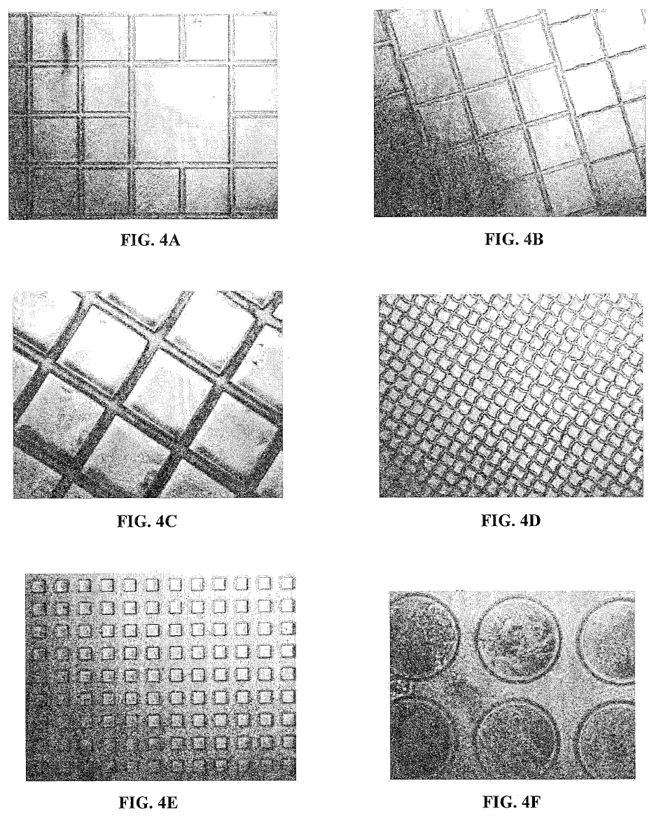

Microwells were fabricated in various dimensions. FIGS. 4A-4F show representative images of additional hydrogel microwell arrays. FIG. 4A shows a 400×400 μm well with a 50 μm-thick wall. FIG. 4B shows 400×400 μm wells with walls of a 20-100 μm thickness gradient. FIG. 4C shows 600×600 μm wells with 100 μm-thick walls. FIG. 4D shows 100×100 μm wells separated by 20 μm-thick walls. FIG. 4E shows 100×100 μm wells separated by 100 μm-thick walls. FIG. 4F shows wells of 400 μm in diameter seeded with human induced pluripotent stem cells (hiPSCs), separated by 100 μm-thick walls.

### Example 2. Culturing and Differentiation of hiPSCs in Microwells in Comparison to Tissue Culture Plastic

Results and Methods

Culturing of Pluripotent Stem Cells

The microwells as fabricated in Example 1 were coated with matrigel in preparation for culturing cells. Small populations of human induced pluripotent stem cells (hiPSCs) were seeded and cultured in microwells. After 4 days of culture on matrigel-coated microwells, cells were stained for pluripotency markers.

Diferentiation of hiPSCs

Activin A was first used for 3 days to differentiate hiPSCs into definitive endoderm. Then the cells were treated with fibroblast growth factor-4 (FGF4) and CHIR99021, a selective inhibitor of flycogen synthase kinase 3 (GSK3), for between day 4 and day 7 of differentiation to be further differentiated into hindgut and to generated spheroids. The spheroids were transferred from microwell and embedded in matrigel, where they matured into organoids.

Results

Immunohistochemical staining and fluorescent microscopy confirmed cultured hiPSCs stained positive for pluripotency markers Oct4 and Sox 2. FIG. 4F shows a bright-field image of hiPSCs seeded in microwells. This result indicated hiPSCs remained pluripotent in microwells. As a comparison, fluorescent microscopic imaging of hiPSCs cultured for 4 days on a silanized glass surface showed cells were spreading and were stained positive for Oct4 and Sox2. Cultured hiPSCs in microwells (300 m×300 μm with 20 μm walls) appeared to have balled up within each well and were also stained positive of Oct4 and Sox2.

Differentiating cells in microwells (300 μm×300 μm with 20 μm walls) was compared with differentiating cells on standard tissue culture plastic. Bright-field microscopic imaging on day 1, day 4, and day 8 after differentiation showed cells aggregated into different sizes and randomly distributed on tissue culture plastic at day 8, whereas cells differentiated in microwells aggregated into spheroid within each well at day 8.

Differentiated cells were stained for a hindgut marker, Cdx2, and a foregut marker, Sox2.

The microwells were demonstrated to be successfully used to culture the smallest-budding unit of intestinal spheroids to better analyze the microenvironment pertinent to spheroid formation and to model the budding process for more reproducible and controllable differentiation. This was believed to shed light on pre-organoid biology and benefit the application of organoids.

We claim:

1. A method of preparing a patterned hydrogel adherent to a solid support comprising
exposing a hydrogel precursor solution to a pattern of light to induce formation of patterned hydrogel and bonding with the solid support,
wherein the solid support is placed on top and in contact with the hydrogel precursor solution for light exposure and the light is applied to the hydrogel precursor solution through the pattern towards the solid support.

2. The method of claim 1, wherein the hydrogel precursor solution is contained in a device secured to a surface where the pattern of light transmits.

3. The method of claim 2, wherein the pattern is formed of a photomask.

4. The method of claim 1, wherein the hydrogel precursor solution comprises a solute with one or more functional groups selected from the group consisting of methacrylates, acrylates, ethylenes, dienes, styrenes, halogenated olefins, vinyl esters, acrylonitriles, acrylamides, and n-vinyls.

5. The method of claim 1, wherein the hydrogel precursor solution comprises a solute selected from the group consisting of poly(ethylene glycol) dimethacrylate, poly(ethylene glycol) diacrylate, tetraethylene glycol diacrylate, ethylene glycol dimethacrylate, ethylene glycol diacrylate, dipropylene glycol dimethacrylate, dipropylene glycol diacrylate, and a polymer or block copolymer thereof.

6. The method of claim 1, wherein the solid support comprises one or more acrylates or methacrylates.

7. The method of claim 1, wherein the solid support is a film, glass, modified silicon, ceramic, or plastic.

8. The method of claim 1, wherein the solid support is a coverslip.

9. The method of claim 2, wherein the device secured to a photomask is made with a photoresist selected from the group consisting of SU-8, poly(methyl methacrylate), poly(methyl glutarimide), phenol formaldehyde resin, and off-stoichiometry thiol-enes (OSTE) polymers.

10. The method of claim 1, wherein the patterned hydrogel is an array of microwells, microchannels, or a combination thereof.

11. The method of claim 1, wherein the patterned hydrogel forms a microfluidic device.

12. The method of claim 1, wherein the patterned hydrogel has a thickness greater than about 20 μm.

13. The method of claim 1, wherein the patterned hydrogel has a thickness between about 20 μm and about 1200 μm.

14. A kit to make a patterned hydrogel adherent to a solid support comprising
a photomask with a pattern of clear and dark regions to allow transmission of a pattern of light,
a device of at least 20 m in height for securing to the photomask
a hydrogel precursor solution comprising one or more solutes capable of being photopolymerized, or the solutes and a diluent in separate containers,
and a solid support comprising one or more acrylate or methacrylate functional groups.

15. The kit of claim 14, wherein the solutes is selected from the group consisting of poly(ethylene glycol) dimethacrylate, poly(ethylene glycol) diacrylate, tetraethylene glycol diacrylate, ethylene glycol dimethacrylate, ethylene glycol diacrylate, dipropylene glycol dimethacrylate, dipropylene glycol diacrylate, and a polymer or block copolymer thereof.

16. The kit of claim 14, wherein the ring is made from a photoresist selected from the group consisting of SU-8, poly(methyl methacrylate), poly(methyl glutarimide), phenol formaldehyde resin, and off-stoichiometry thiol-enes (OSTE) polymers.

17. The kit of claim 14, wherein the solid support is a film, glass, modified silicon, ceramic, or plastic.

18. The kit of claim 14, wherein the solid support is a coverslip.

19. A patterned hydrogel adherent to a solid support comprising
a hydrogel in an array of microwells, microchannels, or a combination thereof,
and a solid support covalently attached to the hydrogel,
wherein the microwells have a depth between about 20 μm and about 1200 μm.

20. The patterned hydrogel of claim 19, wherein the solid support is a film, glass, modified silicon, ceramic, or plastic.
